(19) United States
(12) Patent Application Publication
(10) Pub. No.: US 2014/0212869 A1
(43) Pub. Date: Jul. 31, 2014

(54) Nucleic Acid Proximity Assay Involving the Formation of a Three-way junction

(71) Applicant: AGILENT TECHNOLOGIES, INC., Loveland, CO (US)

(72) Inventors: Nicholas M. Sampas, Santa Clara, CA (US); Robert A. Ach, Santa Clara, CA (US); Nazumi Alice Yamada, Santa Clara, CA (US); Kristin Bernick, Santa Clara, CA (US)

(73) Assignee: AGILENT TECHNOLOGIES, INC., Loveland, CO (US)

(21) Appl. No.: 13/750,810

(22) Filed: Jan. 25, 2013

(51) Int. Cl. C12Q 1/68

(52) U.S. Cl. CPC C12Q 1/6813
USPC 435/6.11

(57) ABSTRACT

Provided herein is a proximity assay that, in certain embodiments, involves: (a) hybridizing a first oligonucleotide and a second oligonucleotide with a target nucleic acid, wherein the first oligonucleotide comprises: i. a region that is complementary to a first sequence in the target nucleic acid and ii. a barcode sequence; and the second oligonucleotide comprises i. a region that is complementary to a second region in the target and ii. the complement of the barcode sequence; and (b) detecting hybridization between the barcode sequence and the complement of the barcode sequence, wherein hybridization between the barcode sequence and the complement of the barcode sequence indicates that the first and second target sequences are proximal to one another in the sample.

### BACKGROUND

Measuring the proximity of two nucleic acid sequences is useful for determining the status of genomic structural variations as well as the primary structure of gene transcripts, for example by splice variance. Described herein is an assay for the detection and localization of nucleic acids that are in close proximity.

### SUMMARY

Provided herein is a proximity assay that, in certain embodiments, involves: (a) hybridizing a first oligonucleotide and a second oligonucleotide with a target nucleic acid, wherein the first oligonucleotide comprises: i. a region that is complementary to a first sequence in the target nucleic acid and ii. a barcode sequence; and the second oligonucleotide comprises i. a region that is complementary to a second region in the target and ii. the complement of the barcode sequence; and (b) detecting hybridization between the barcode sequence and the complement of the barcode sequence, wherein hybridization between the barcode sequence and the complement of the barcode sequence indicates that the first and second target sequences are proximal to one another in the sample.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates one embodiment of the subject method.

FIG. 2 illustrates one way in which hybridization can be detected.

FIG. 3 illustrates another way in which hybridization can be detected.

FIG. 4 illustrates an embodiment of the method that involves a hairpin.

FIG. 5 illustrates an embodiment of the method that uses short oligonucleotides as quenchers.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length, for example.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42 C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “in situ” refers to “inside a cell”. For example, the RNA being detected by in situ hybridization is present inside a cell. The cell may be permeabilized or fixed, for example.

The term “contacting” means to bring or put together. As such, a first item is contacted with a second item when the two items are brought or put together, e.g., by touching them to each other or combining them in the same solution.

The term “in situ hybridization conditions” as used herein refers to conditions that allow hybridization of a nucleic acid to a complementary nucleic acid, e.g., a sequence of nucleotides in a RNA or DNA molecule and a complementary oligonucleotide, in a cell. Suitable in situ hybridization conditions may include both hybridization conditions and optional wash conditions, which conditions include temperature, concentration of denaturing reagents, salts, incubation time, etc. Such conditions are known in the art.

The term “duplex,” or “duplexed,” as used herein, describes two complementary polynucleotides that are base-paired, i.e., hybridized together.

As used herein, the term “Tₘ” refers to the melting temperature of an oligonucleotide duplex at which half of the duplexes remain hybridized and half of the duplexes dissociate into single strands. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

The term “free in solution,” as used here, describes a molecule, such as a polynucleotide, that is not bound or tethered to another molecule.

The term “ligating”, as used herein, refers to the enzymatically catalyzed joining of the terminal nucleotide at the 5′ end of a first DNA molecule to the terminal nucleotide at the 3′ end of a second DNA molecule.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

If two nucleic acids are “complementary”, they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

The term “digesting” is intended to indicate a process by which a nucleic acid is cleaved by a restriction enzyme. In order to digest a nucleic acid, a restriction enzyme and a nucleic acid containing a recognition site for the restriction enzyme are contacted under conditions suitable for the restriction enzyme to work. Conditions suitable for activity of commercially available restriction enzymes are known, and supplied with those enzymes upon purchase.

A “oligonucleotide binding site” refers to a site to which an oligonucleotide hybridizes in a target polynucleotide. If an oligonucleotide “provides” a binding site for a primer, then the primer may hybridize to that oligonucleotide or its complement.

In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “unique sequence”, as used herein, refers to nucleotide sequences that are different one another, or their complements. For example, a first unique sequence has a different nucleotide sequence than a second unique sequence or its complement. Unless otherwise indicated, a unique sequence is only present in one polynucleotide in a sample.

The term “do not hybridize to each other”, as used herein in the context of nucleic acids that do not hybridize to each other, refers to sequences that been designed so that they do not anneal to one another under stringent conditions.

The term “similar to one another” in the context of a polynucleotide or polypeptide, means sequences that are at least 70% identical, at least 80% identical, at least 90% identical, or at least 95% identical, to one another.

Other definitions of terms may appear throughout the specification.

### Description of Exemplary Embodiments

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

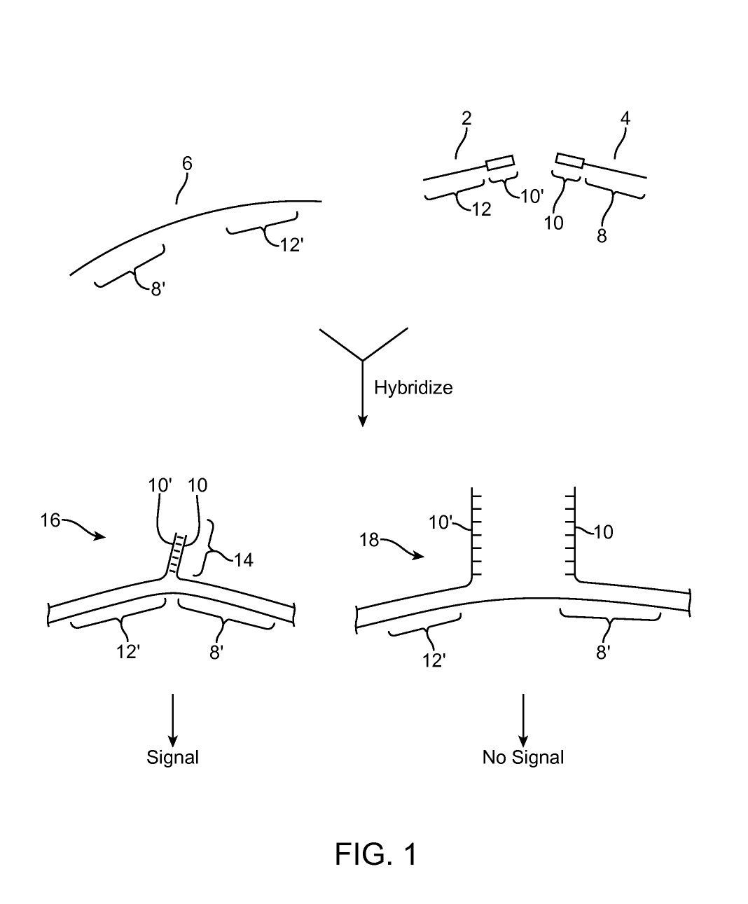

With reference to FIG. 1, one embodiment of the method comprises hybridizing a first oligonucleotide 2 and a second oligonucleotide 4 with a target nucleic acid 6. As illustrated, the first oligonucleotide may comprise a region 8 that is complementary to a first sequence 8′ in the target nucleic acid and a barcode sequence 10. The second oligonucleotide may comprise a region 12 that is complementary to a second region 12′ in the target nucleic acid and the complement of the barcode sequence 10′. The method further comprises detecting hybridization between the barcode sequence 10 and the complement of the barcode sequence 10′ to produce duplex 14. When the first and second target sequences are proximal to one another in the target nucleic (as illustrated by complex 16 in FIG. 1), barcode sequence 10 and its complement 10′ hybridize to produce duplex 14 that can be detected. Detection of duplex 14 indicates that the first and second target sequences are proximal to one another in the target nucleic. When the first and second target sequences are distal to one another in the target nucleic (as illustrated by complex 18 in FIG. 1), barcode sequence 10 and its complement 10′ do not hybridize and, as such, no duplex is produced.

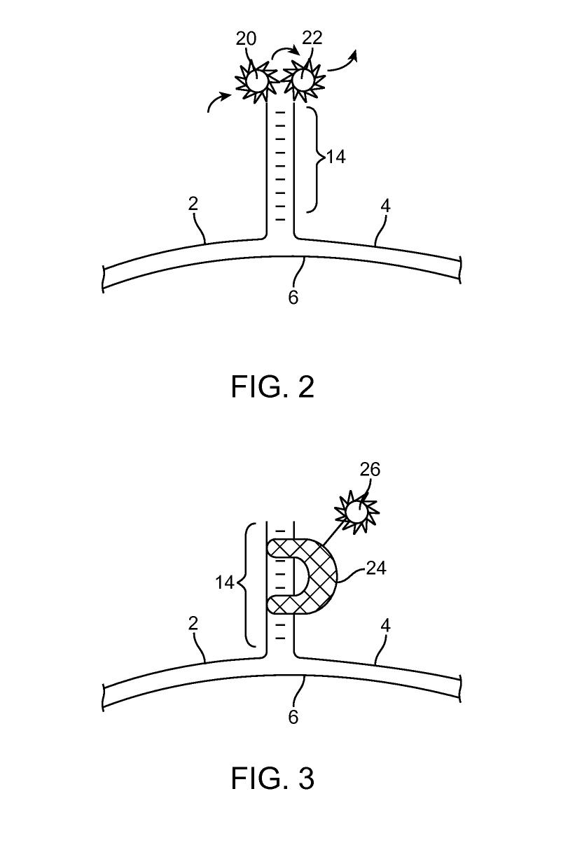

Duplex 14 can be detected in a variety of different ways. For example, as illustrated in FIG. 2, the first oligonucleotide 2 may be labeled with a first fluorophore 20 and the second oligonucleotide 4 may be labeled with a second fluorophore 22, where the first the second fluorophores provide a fluorescence resonance energy transfer (FRET) signal when the barcode sequence 10 and its complement 10′ are hybridized to one another. The detection step of the method may involve detecting the FRET signal, thereby indicating the first and second target sequences are proximal to one another in the target nucleic acid.

In other embodiments, the duplex may be detected using a sequence-specific nucleic acid binding protein that binds to duplex 14. In these embodiments, the sequence-specific nucleic acid binding protein may comprise a nucleic acid binding domain from a nucleic acid binding protein such as a transcription factor, or a restriction endonuclease. Given that the sequence to which several DNA binding proteins bind have been defined, one can design the barcode sequence 10 so that it binds to a known DNA domain, e.g., a helix-turn-helix, zinc finger, leucine zipper, winged helix, winged helix turn helix, helix-loop-helix, HMG-box domain. Alternatively, the sequence-specific DNA binding protein used may be engineered to bind to the barcode. Zinc finger proteins and TAL effector proteins can be engineered to bind to virtually any sequence (see, e.g., Pabo et al Annual Review of Biochemistry 2001 70: 313-340; Jamieson Nature Reviews Drug Discovery 2003 2: 361-368; Boch et al Science 2009 326: 1509-12; Morbitzer et al. Proceedings of the National Academy of Sciences 2010 107: 21617-21622; and Miller et al Nature Biotechnology 2010 29: 143) and, as such, may be used in this embodiment of the method. In a particular embodiment, the sequence-specific DNA binding protein used may be a cleavage-deficient restriction endonuclease, methods for the production of which are known (see, e.g., Dorner et al Nucleic Acids Res. 1994 22:1068-74 and Xu et al Biotechniques 1993 15:310-5). As would be readily apparent, if a nucleic acid binding protein is used, it may be exogenous to the cell being analyzed.

In some embodiments, the sequencing-specific nucleic acid binding protein may be unlabeled, and the nucleic acid binding protein may be detected using an antibody that can be conjugated with an optically detectable label or an enzyme that catalyzes the synthesis of a chromogenic compound that can be detected visually or using an imaging system. In one embodiment, horseradish peroxidase (HRP) may be used, which can convert chromogenic substrates (e.g., TMB, DAB, or ABTS) into colored products, or, alternatively, produce a luminescent product when chemiluminescent substrates (e.g. ECL) are used. In certain cases, the antibody may be conformation-specific in that it only binds to the DNA binding protein after the protein binds to the duplex.

In some cases, the sequencing-specific nucleic acid binding protein may be labeled, e.g., with an optically detectable label, e.g., a fluorophore or with a chromogenic enzyme as discussed above. In certain embodiments and as illustrated in FIG. 3, the method may comprise binding DNA binding protein 24 that is conjugated to a label 26 to the duplex, and then directly detecting the label of the protein. In other embodiments, one of the first or second oligonucleotides may be labeled with a second fluorophore, and the first fluorophore (i.e., the one on the DNA binding protein protein) and the second fluorophore may provide a fluorescence resonance energy transfer (FRET) signal when the sequencing-specific nucleic acid binding protein is bound to the duplex.

In other embodiments, the first oligonucleotide may be labeled with a first fluorophore and the second oligonucleotide may be labeled with a quencher of the first fluorophore, and the quencher can be cleaved from the second oligonucleotide by an enzyme (e.g., a restriction enzyme or CAS endonuclease) that binds to the duplex 14, thereby activating the first fluorophore. This step of the method could be done using a restriction enzyme or, in alternative cases, could be done using an engineered endonuclease (e.g., a TAL effector fused to the cleavage domain of FokI to create a TAL effector nuclease, i.e., a “TALEN”, as described in, e.g., Christian et al (Genetics 186 2010: 757-61) and (Li et al, Nucleic Acids Res 2010 39: 359-72)). In certain cases, the cleavage can be done using a wild type or variant CAS endonuclease that binds to a CRISPR stem loop e.g., a CAS6 endonuclease. Wild type CAS6 proteins and corresponding CRISPR stem loops are part of the CRISPR-CAS adaptive immune system found in many bacteria and archaea. The CRISPR-CAS system is reviewed in a number of publications, including Sternberg et al (RNA 2012 18: 661-72), Makarova et al (Biol Direct. 2011 6: 38), Deltcheva et al (Nature 2011 471: 602-7), Wang et al (Structure 2011 19: 257-64), Carte et al (RNA 2010 16: 2181-8), Carte et al (Genes Dev. 2008 22: 3489-96), and Haurwitz et al (Science. 2010 329: 1355-8), which are incorporated by reference. A CAS6 protein may be catalytically active in that it catalyzes the cleavage of a CRISPR stem loop. Certain embodiments of the method may employ a CAS6 protein that is not catalytically active.

The designation of “proximal” and “distal” depends on how the method is implemented and the lengths of any linkers, etc., that are used to tether the fluorophores and/or if there are nucleotides between the sequences that are complementary to the target nucleic acid (i.e., 8 and 12) and the barcode and complement (10 and 10′). If FRET is used, the fluorophores should be within 10 nanometers of each other so that FRET can occur. In particular embodiments, the term “proximal” means that the sequences are linked by less than 20 nucleotides, e.g., less than 10 or less than 5 nucleotides. In certain embodiments, “distal” sequences may be at least 20 nucleotides apart, e.g., at least 50, at least 100, at least 500, or at least 1,000 nucleotides apart. In particular cases, distal sequences may be on different chromosome arms or in certain cases may be unlinked, e.g., on different nucleic acid molecules or different chromosomes.

The nucleotide sequence of the barcode should be unique in the sense that it does not significantly hybridize to any other sequences in the sample. Further, when the barcode sequences are used in a multiplex manner, they should not hybridize with one another (except for its complement) and they should be Tm-matched, where the term “Tₘ-matched” refers to a set of oligonucleotides that have Tₘs that are within a defined range, e.g., within 5° C. or 10° C. of one another. Sets of non-cross-hybridizing sequences are described in, e.g., US20070259357, US20030077607, US20100311957, and Brenner et al (Proc. Natl. Acad. Sci. 1992 89:5381-3). Further, computer algorithms for selecting non-crosshybridizing sets of sequences are described in Brenner (PCT Publications No. WO 96/12014 and WO 96/41011) and Shoemaker (Shoemaker et al., European Pub. No. EP 799897 A1 (1997)). Typically, a segment of unique sequence is from 10 to 60 bases in length, e.g., 10 to 30 bases in length. In particular embodiments, the barcode sequence may be from 5 to 25 bases in length. In certain cases, the barcode sequence and its complement may have a Tm in the range of 60° C. to 80° C.

The region that is complementary to the first sequence in the target nucleic acid and the region that is complementary to the second sequence in the target nucleic acid may be, independently, from 15 to 100 bases in length, although any sequence that is greater than 18 nucleotides in length (e.g., 18 nt to 200 nt) may be used in certain circumstances. In certain cases, the Tₘ of these sequences can be at least 10° C. or at least 15° C. higher than the Tm of the barcode sequences. The relative positioning of the barcode, the complement of the barcode and the other regions in the oligonucleotides may vary greatly. For example, the barcode, the complement of the barcode and the target-complementary regions may be, independently, at the 3′ end, the 5′ end, or in the middle of the oligonucleotides. The arrangement of elements shown in the figures is merely an example of an arrangement.

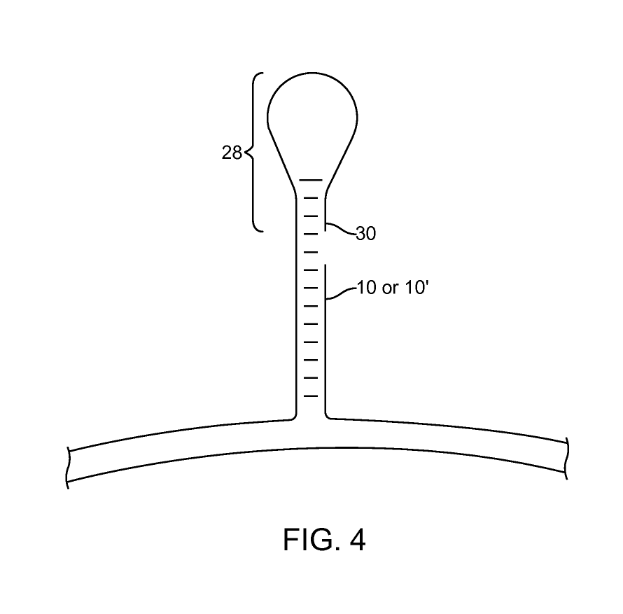

In particular embodiments and with reference to FIG. 4 one of the first or second oligonucleotides may comprise a hairpin 28. In some embodiments and as illustrated in FIG. 4, the terminal nucleotide 30 at the recessed end of hairpin 28 may be immediately adjacent to the barcode sequence or the complement of the barcode sequence when the barcode sequence and the complement of the barcode sequence are hybridized. In such a complex, the hairpin region promotes a phenomenon termed stacking (which phenomenon may also be called coaxial stacking) which allows the polynucleotide to bind more tightly, i.e., more stably. In effect, in this embodiment, the duplex produced by binding of the two oligonucleotides to a target nucleic acid resembles a long hairpin structure containing a nick in the stem of the hairpin. Stacking and its effect on duplex stability are discussed in Liu et al (Nanobiology 1999; 4: 257-262), Walter et al (Proc. Natl. Acad. Sci. 1994 91:9218-9222) and Schneider et al (J. Biomol. Struct. Dyn. 2000 18:345-52), as well as many other references. In these embodiments, the method further comprises ligating the first and second oligonucleotides to each other. In certain cases, ligation may regenerate a restriction site that can be cleaved using a suitable endonuclease.

In certain embodiments, one of the first and second oligonucleotides may be immobilized on a solid support, e.g., in the form of an array. In this embodiment, the hybridization and detection may in a reaction that occurs on the surface of an array substrate.

The targeted nucleic acid may be any type of nucleic acid, including genomic DNA, RNA (including unprocessed RNA and processed RNA) or cDNA. In certain cases, the hybridizing may be done in vitro on an isolated target nucleic acid. In other embodiments, the hybridizing may be done in situ and the target nucleic acid may be an intact chromosome or RNA.

In some cases, the hybridizing may be done in situ and the target nucleic acid is in a living cell (see, e.g., Wiegang et al Methods Mol. Biol. 2010 659:239-46; Dirks et al Methods 2003 29: 51-7; and Lorenz RNA 2009 15:97-103).

Certain hybridization methods used herein include the steps of fixing a biological or non-biological sample (e.g., intact chromosomes or cells), hybridizing oligonucleotides to RNA or DNA molecules (e.g., RNAs or chromosomes) contained within the fixed sample, and washing the hybridized sample to remove non-specific binding. In situ hybridization assays and methods for sample preparation are well known to those of skill in the art and need not be described in detail here. Such methods can be found in, for example, Amann R. et al., 1995, Microbiol. Rev. 59(1): 143-69; Bruns and Berthe-Corti, 1998, Microbiology 144, 2783-2790; Vesey G. et al., 1998, J. App. Microbiol. 85, 429-440; and Wallner G. et al., 1995, Appl. Environ. Microbiol. 61(5): 1859-1866, and US20100081131, which are incorporated by reference herein.

Permeabilized/fixed cells are contacted with labeled polynucleotides under in situ hybridizing conditions, where “in situ hybridizing conditions” are conditions that facilitate annealing between a nucleic acid and the complementary nucleic acid. Hybridization conditions vary, depending on the concentrations, base compositions, complexities, and lengths of the probes, as well as salt concentrations, temperatures, and length of incubation. For example, in situ hybridizations typically are performed in hybridization buffer containing 1-2×SSC, 50% formamide, and blocking DNA to suppress non-specific hybridization. In general, hybridization conditions include temperatures of about 25° C. to about 55° C., and incubation times of about 0.5 hours to about 96 hours. Suitable hybridization conditions for a library of oligonucleotides and target microbe can be determined via experimentation which is routine for one of skill in the art.

Certain fluorescence in situ hybridization (FISH) methods offer many advantages over radioactive and chromogenic methods for detecting hybridization. Not only are fluorescence techniques fast and precise, they allow for simultaneous analysis of multiple signals that may be spatially overlapping. Through use of appropriate optical filters, it is possible to distinguish multiple different fluorescent signals in a single sample using their excitation and emission properties alone. Methods for combinatorial labeling are described in, e.g., see, Ried et al., 1992, Proc. Natl. Acad. Sci. USA 89, 1388-1392; Tanke, H. J. et al, 1999, Eur. J. Hum. Genet. 7: 2-11. By using combined binary ratio labeling (COBRA) in conjunction with highly discriminating optical filters and appropriate software, over 40 signals can be distinguished in the same sample, see, e.g., Wiegant J. et al., 2000, Genome Research, 10 (6), 861-865.

In certain embodiments, cells are harvested from a biological or non-biological sample using standard techniques. For example, cells can be harvested by centrifuging a sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution. Methods for fixing cells are known in the art and can be adapted to suit different types of microbes, if needed. Determination of suitable fixation/permeabilization protocols are carried out routinely in the art.

A hybridized sample can be read using a variety of different techniques, e.g., by microscopy, such as light microscopy, fluorescent microscopy or confocal microscopy. In embodiments in which oligonucleotides are labeled with a fluorescent moiety, reading of the contacted sample to detect hybridization of labeled oligonucleotides may be carried out by fluorescence microscopy. Fluorescent microscopy or confocal microscopy used in conjunction with fluorescent microscopy has an added advantage of distinguishing multiple labels even when the labels overlap spatially. Methods of reading fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

In one embodiment, an interphase or metaphase chromosome preparation may be produced. The chromosomes are attached to a substrate, e.g., glass, contacted with the probe and incubated under hybridization conditions. Wash steps remove all unhybridized or partially-hybridized probes, and the results are visualized and quantified using a microscope that is capable of exciting the dye and recording images.

Such methods are generally known in the art and may be readily adapted for use herein. For example, the following references discuss chromosome hybridization: Ried et al., Human Molecular Genetics, Vol 7, 1619-1626; Speicher et al, Nature Genetics, 12, 368-376, 1996; Schröck et al., Science, 494-497, 1996; Griffin et al., Cytogenet Genome Res. 2007;118(2-4):148-56; Peschka et al., Prenat Diagn., 1999, December; 19(12): 1143-9; Hilgenfeld et al, Curr Top Microbiol Immunol., 1999, 246: 169-74.

In certain embodiments, the signal obtained from performing the method may be compared with that of a reference sample, e.g., a cell chromosome a healthy or wild-type organism. Briefly, the method comprises contacting under in situ hybridization conditions a test sample with a plurality of probes described above and contacting under in situ hybridization conditions a reference chromosome with the same plurality probes. After hybridization, the emission spectra created from the unique binding patterns from the test sample are compared against those of the reference sample.

In one embodiment, the structure of a test chromosome may be determined by comparing the pattern of binding of the probes to the test chromosome with the binding pattern of the same probes with a reference chromosome. The binding pattern of the reference chromosome may be determined before, after or at the same time as the binding pattern for the test chromosome. This determination may be carried out either manually or in an automated system. The signal associated with the test chromosome can be compared to the binding pattern that would be expect for known deletions, insertions, translocation, fragile sites and other more complex rearrangements, and/or refined breakpoints. The matching may be performed by using computer-based analysis software known in the art. Determination of identity may be done manually (e.g., by viewing the data and comparing the signatures by hand), automatically (e.g., by employing data analysis software configured specifically to match optically detectable signature), or a combination thereof.

In another embodiment, the test sample is from an organism suspected to have cancer and the reference sample may comprise a negative control (non-cancerous) representing wild-type genomes and second test sample (or a positive control) representing a cancer associated with a known chromosomal rearrangement. In this embodiment, comparison of all these samples with each other using the subject method may reveal not only if the test sample yields a result that is different from the wild-type genome but also if the test sample may have the same or similar genomic rearrangements as another cancer test sample.

Proposed herein is a method whereby a first DNA target sequence is hybridized with two oligonucleotide probe sequences. Each of these probe sequences has two domains, one which is complementary to a sequence within the target sequence and another, hereby referred to as a “barcode sequence”, which is not contained within the target sequence. The target-specific domain of each probe is hereby referred to as the “test sequence”, and the two test sequences can be substantially adjacent to each other where they specifically bind to the target sequence. The barcode sequences of the two probes are substantially complementary to each other and can reside at opposite ends of the probe sequences, such that the barcode sequence at the 3′ end of a first probe is complimentary to the barcode sequence at the 5′ end of the second probe. Thus, if the two complementary barcode sequences are in close proximity due to the hybridization of the two test sequences to the adjacent regions of the target sequence, then the barcode sequences will hybridize to each other forming a 3-way DNA Junction (FIG. 1).

In some embodiments, the method may be used to determine the proximity of two sequences. In one embodiment, the ends of the barcode sequences are labeled with donor and acceptor dye molecules (or particles) so that the complex (or assembly) will exhibit FRET (Fluorescence Resonant Energy Transfer; see Santangelo at al Nuc. Acids Res. 32, 6, e57 2004). In an alternative embodiment, the two ends could also be labeled with a fluorescent dye and a quencher, so that binding of both probe sequences resulting in a suppression of the fluorescent signal, whereas binding of only the fluorescent probe would still result in fluorescence. This latter embodiment may benefit by the use of a control in which each labeled oligo probe is exposed to the sample in the absence of the quencher counterpart.

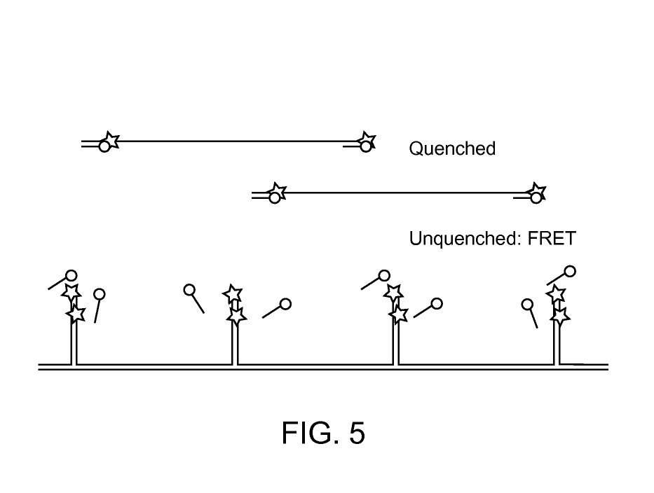

In one embodiment, the first oligonucleotide may be labeled with a fluorophore and may comprise a quencher oligonucleotide that is base paired with the barcode sequence of the first oligonucleotide. In this embodiment, the quencher oligonucleotide comprises a quencher that quenches the fluorophore and the quencher oligonucleotide is displaced by the complement of the barcode sequence in the second oligonucleotide to unquench the fluorophore. This should allow hybridization between the barcode sequence and the complement of the barcode sequence to be detected, either through direct detection of a fluorophore, or by detecting a FRET signal. Certain aspects of this embodiment are shown in FIG. 5. In this embodiment, the probes are quenched by short oligonucleotides containing quencher molecules and are in the “off” state. In the presence of target, the barcodes base pair with one another and the quencher oligonucleotides are displaced, thereby turning the probes “on”. In the embodiment shown, short oligonucleotides containing quenchers at one end are designed to hybridize to the barcode regions of the oligonucleotide probes in such a way that the quencher is in close proximity (<5 nm) to the fluorophore. This results in quenching the fluorophores in the absence of target. When the probes bind their target (RNA or DNA), the barcodes form a more stable structure that displaces the shorter oligonucleotides resulting in the fluorophores being turned “on”. In certain cases, the quencher oligonucleotide can be mixed with the probe oligonucleotides, allowed to hybridize, and then hybridized to its target in quenched form. If the oligonucleotides are to be delivered to a living cell, then cell penetrating peptides, toxin-mediated cell membrane permeabilization (using, e.g., Streptolysin O), microinjection or electroporation could be used.

Another embodiment involves the binding of a protein, antibody, aptamer, or complex that specifically recognizes the double-stranded barcode sequence. One example of such a protein is a zinc-finger protein. Zinc fingers can sequence-specifically recognize three DNA bases, and these can be assembled combinatorially to generate longer DNA sequence recognition motifs. The binding of a protein to the triplex may also help to stabilize it. Furthermore, the labeling of the protein component would provide a localized signal that indicates the binding of the probe-probe-target triplex. The binding protein itself may be fluorescently labeled or fused to known fluorescent protein motifs, such as GFP; secondary detection by specific labeled antibodies that target the binding protein is also disclosed here and may act as a means of signal amplification, whereby multiple secondary antibodies could be bound to the barcode-binding protein. Alternatively, a FRET signal could be produced if any two (or more) of the 3 components (2 probes and the binding protein) are labeled. Proteins that can recognize DNA sequences include regulatory proteins which have the following sequence recognizing motifs: zinc-finger motif, leucine zipper motif, the helix-turn-helix motif, or Transcription Activator Like Effector (TALE) motifs, for example.

In general terms, the barcode sequences should be sufficiently long to add a small degree of stability to the duplexes formed between the probes and target relative to the binding affinity of test sequence. However, the barcode sequences should be sufficiently short to inhibit base-pairing between the probes in the absence of the target. Thus, in certain cases, the useful lengths of the barcode sequences are between 5 bases and 25 bases and the useful lengths of the probe test sequences are 20 bases or longer. In certain cases, typical oligonucleotide sequences complementary to the target DNA in a FISH assay are 50-150 base pairs long. Shorter sequences (20-100 bp) may be used to target RNA sequences, such as messenger RNAs. For the assay described here to be most informative, the complementary regions of the two barcode (“twist-tie”) sequences can be sufficiently long to hybridize to each other after the test sequence has hybridized and should add somewhat to the stability of the duplex between each probe and target pair, but they should be short enough to preclude appreciable hybridization in the absence of target binding.

In one embodiment, the method can be multiplexed in the form of a DNA microarray assay in which many thousands of breakpoints can be simultaneously interrogated. In a microarray assay, each surface-bound oligonucleotide may serves as one of the probes and the second oligonucleotide may be spiked into the hybridization mix along with the target genomic sequences, all of which are exposed to the array for co-hybridization. For targets containing a sequence that is largely complementary to the array-bound probe sequence and for which that section is flanked by a sequence complementary to the solution phase probe sequence (plus the reporter sequence), the double-stranded 3-way junction can be probed by a labeled sequence specific protein targeting the barcode sequence. In this assay, the barcodes can all be the same and the spatial information of the probe on the array tells what sequence pairs are being targeted. Again, this assay can be done either by direct fluorescence of labels bound to the barcode-duplex, or by FRET involving two or more labels attached to the probes or the protein.

In another embodiment, the method may make use of two dissimilar barcode sequences on each of the two probes. These sequences, though still partially complementary, differ in length. Specifically, one of the barcode sequences contains a portion of a hairpin structure that includes one stem region as well as a hairpin loop and another partial stem region. And, the other barcode contains a missing portion of that second stem structure. When hybridized under the right conditions, these probes create a hairpin structure with a single-strand break in the stem. This break can be ligated by means of an enzymatic ligase reaction, thus chemically bonding the two probes. The ligation can serve three purposes: first, it stabilizes the hybridization of the probes to the target sequence by doubling the length of the duplex; second, the ligation stabilizes and further ensure the presence of the hairpin for recognition by the hairpin-binding protein; and, third, the hairpin created can be detected by an even larger set of proteins.

In one embodiment, the hairpin formed by hybridization of the barcode sequences forms an aptamer known to bind a specific protein. This embodiment greatly expands the number of proteins (with an increased set of properties) that can be used for detection. Aptamers can also be designed to bind molecules other than proteins, which again greatly increases the utility of the assay. For example, an aptamer which specifically bound a fluorescent dye molecule could be used.

In these embodiments, it should be noted that when properly designed, the stem loop should not form in the absence of binding both probes to the same target molecule. Thus the formation of the stem indicates the proximity of those sequences on the same molecule. In another embodiment, the presence of the hairpin can be tested by means of antibodies or any other protein or complex known to bind anywhere to that structure. A protein that specifically recognized the barcode hairpin would specifically bind only to barcodes bound next to each other on the target.

Oligonucleotide FISH (“oFISH”) is typically done, not with a single oligo, but with a number of distinct oligonucleotides (typically more than 50 for DNA FISH, and typically more than 10 for RNA FISH), typically spanning a substantial fraction of a genomic interval with multiple adjacent oligonucleotides probes. This method, as described above, generates a single nucleic acid-triplex, and may involve a single reporter molecule. Likewise, the methods described above can be applied analogously to oligo-FISH with a multitude of oligonucleotides, but where the specific test sequences map to one or more contiguous genomic or RNA sequences and where an increased signal is produced by having multiple labeled oligonucleotides hybridized. In this implementation the barcode sequences may all have the same complementary sequence, or they could differ so that each barcode sequence is complementary to one of its adjacent neighbors. Additionally, the probe sequences can be designed so that each probe contains barcode sequences at both its 5′ and 3′ ends, with each being complimentary to the barcodes of the adjacent probes as they are flanked on the target sequence. In this way the probes form a daisy chain of sequences each attached to the next in a series along the targeted interval. In this latter implementation, the barcode sequences help to ensure that the oligos hybridize to each other in a specific order (i.e. if each barcode duplex is distinct). Also, depending on the degeneracies of the binding protein, they can be designed to be specific targets for a common DNA-binding protein. It is possible that if a binding protein is introduced during or after the hybridization that whole duplex assembly will be further stabilized.

In addition to the above applications, this system may be exploited to detect mRNA splice variants. Each barcoding region may be designated to a specific fluorescent color, whereby the combination of colors per mRNA molecule detected could be used to determine splice variants. Since each barcode can indicate the presence of one detection agent, multiplexed detection is readily possible to differentiate between different splice variants.

In certain embodiments, oligonucleotide probes may be designed using methods set forth in US20040101846, U.S. Pat. No. 6,251,588, US20060115822, US20070100563, US20080027655, US20050282174, patent application Ser. No. 11/729,505, filed March 2007 and patent application Ser. No. 11/888,059, filed Jul. 30, 2007 and references cited therein, for example. In certain embodiments, the oligonucleotides may be synthesized in an array using in situ synthesis methods in which nucleotide monomers are sequentially added to a growing nucleotide chain that is attached to a solid support in the form of an array. Such in situ fabrication methods include those described in U.S. Pat. Nos. 5,449,754 and 6,180,351 as well as published PCT application no. WO 98/41531, the references cited therein, and in a variety of other publications. In one embodiment, the oligonucleotide composition may be made by fabricating an array of the oligonucleotides using in situ synthesis methods, and cleaving oligonucleotides from the array. The oligonucleotides may be amplified prior to use (e.g., by using PCR using primer sites that are at the terminal regions of the oligonucleotides, or by using polymerase promoter, e.g., a T7 polymerase promoter, that is at a terminal region of the oligonucleotides).

In some cases, multiple oligonucleotide probes may be manufactured by PCR of a mixed template using a pair of primers. In these cases, it would be convenient if the primer sequence contains the barcode sequence so that the primers do not need to be cleaved from the oligonucleotides before use. However, complementary sequences are not useful for primers as they will form primer dimers during PCR. An approach that eliminates this problem is to use alternating pairs of primers for probes. So, if we consider numbering probes along a genomic interval sequentially, then the odd probes would have two primers with unrelated sequences, A and B. And, the even probes would have primers with sequences complementary to the odd primers, B′ and A′. In this way there would be two sets of barcode duplexes, AA′ and BB′.

### Kits

Also provided by this disclosure is a kit for practicing the subject method, as described above. The various components of the kit may be present in separate containers or certain compatible components may be pre-combined into a single container, as desired.

In addition to above-mentioned components, the subject kits may further include instructions for using the components of the kit to practice the subject methods, i.e., to provide instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

1. A method comprising:
(a) hybridizing a first oligonucleotide and a second oligonucleotide with a target nucleic acid, wherein:
said first oligonucleotide comprises: i. a region that is complementary to a first sequence in said target nucleic acid and ii. a barcode sequence; and
said second oligonucleotide comprises i. a region that is complementary to a second region in said target nucleic acid and ii. the complement of said barcode sequence; and
(b) detecting hybridization between said barcode sequence and the complement of said barcode sequence, wherein hybridization between said barcode sequence and the complement of said barcode sequence indicates that said first and second target sequences are proximal to one another in said target nucleic acid.

2. The method of claim 1, wherein:
said first oligonucleotide is labeled with a first fluorophore and said second oligonucleotide is labeled with a second fluorophore, and said first and said second fluorophores provide a fluorescence resonance energy transfer (FRET) signal when said barcode sequence and the complement of said barcode sequence are hybridized to one another; and
said detecting step (b) detects said FRET signal.

3. The method of claim 1, wherein said detecting step (b) is done using a sequence-specific nucleic acid binding protein that binds to the duplex produced by hybridizing said barcode sequence and the complement of said barcode sequence.

4. The method of claim 3, wherein said sequence-specific nucleic acid binding protein comprises a DNA binding domain from a transcription factor.

5. The method of claim 3, wherein said sequence-specific nucleic acid binding protein is a CRISPR endonuclease.

6. The method of claim 3, wherein said sequence-specific nucleic acid binding protein is a cleavage-deficient restriction endonuclease.

7. The method of claim 3, wherein said sequencing-specific nucleic acid binding protein is labeled with a first fluorophore.

8. The method of claim 7, wherein at least one of said first or second oligonucleotides is labeled with a second fluorophore, and said first fluorophore and said second fluorophore provide a fluorescence resonance energy transfer (FRET) signal when said sequencing-specific DNA binding protein is bound to said duplex.

9. The method of claim 3, wherein binding of said sequence specific nucleic acid binding protein is detected using a labeled antibody.

10. The method of claim 1, wherein said first oligonucleotide is labeled with a first fluorophore and said second oligonucleotide is labeled with a quencher of said first fluorophore, and said quencher is cleaved from the second oligonucleotide by a restriction enzyme that binds to the duplex produced by hybridizing said barcode sequence and the complement of said barcode sequence, thereby activating said first fluorophore.

11. The method of claim 1, wherein said barcode sequence is from 5 to 25 bases in length.

12. The method of claim 11, wherein said first oligonucleotide is labeled with a fluorophore and comprises a quencher oligonucleotide that is base paired with said barcode sequence, wherein said quencher oligonucleotide comprises a quencher that quenches said fluorophore and said quencher oligonucleotide is displaced by said complement of said barcode sequence in said second oligonucleotide to unquench said fluorophore and allow hybridization between said barcode sequence and the complement of said barcode sequence to be detected.

13. The method of claim 1, wherein said first or second oligonucleotides comprise a hairpin.

14. The method of claim 13, wherein the terminal nucleotide at the recessed end of said hairpin is immediately adjacent to the barcode sequence or the complement of said barcode sequence when said barcode sequence and the complement of said barcode sequence are hybridized.

15. The method of claim 14, wherein the method further comprises ligating the first and second oligonucleotides to each other.

16. The method of claim 1, wherein said target nucleic acid is genomic DNA or RNA.

17. The method of claim 1, wherein one of the first and second oligonucleotides is immobilized on a solid support.

18. The method of claim 1, wherein said hybridizing is done in vitro on an isolated target nucleic acid.

19. The method of claim 1, wherein said hybridizing is done in situ and said target nucleic acid is an intact chromosome.

20. The method of claim 19, wherein said hybridizing is done in situ and said target nucleic acid is in a living cell.
